(19) United States
(12) Patent Application Publication
(10) Pub. No.: US 2007/0111217 A1
(43) Pub. Date: May 17, 2007

(54) Quantitative trait locus prognostic for changes in regional adiposity and BMI in Caucasian males

(76) Inventors: Julieta Uthurralt, Washington, DC (US); Heather Gordish-Dressman, Baltimore, MD (US); Eric P. Hoffman, Kensington, MD (US); Joseph M. Devaney, Silver Spring, MD (US)

Correspondence Address: DR. MELVIN BLECHER, 4329 VAN NESS ST., NW, WASHINGTON, DC, 20016, US

(21) Appl. No.: 11/271,918

(22) Filed: Nov. 14, 2005

(51) Int. Cl. C12Q 1/68

(52) U.S. Cl. 435/6

(57) ABSTRACT

Caucasian males, but not females, presenting with statistically significant increases of L162V allelic SNP of the PPARα gene, relative to that of wild-type homozygotes, have an increased likelihood of increased regional adiposity (subcutaneous fat) and BMI, and are more likely to develop diseases associated with such changes, such as type 2 diabetes and metabolic syndrome.

This research was supported by grants from the National Institutes of Health (R01 NS40608 (co-support by NINDS, NIAMS and NIAJ). The United States Government therefore has an interest in this invention.

### FIELD OF THE INVENTION

In general the invention deals with genetic associations of adiposity in humans. More specifically, the invention involves the use of Quantitative Trait Loci (QTL) in the prognosis of regional obesity in adult males.

### BACKGROUND

The incidence of obesity has increased dramatically in the past 20 years affecting both adult and pediatric populations. Excessive fat accumulation is strongly linked to type 2 diabetes, metabolic syndrome, cardiovascular disease and cancer among other disorders. Indeed, obesity and type 2 diabetes are widely acknowledged as an emerging world-wide public health concern. The localization of fat is important, with metabolic syndrome best correlated with abdominal fat accumulation (visceral and/or subcutaneous), and not limb subcutaneous fat. However, age is a major variable in such studies, as central adiposity increases with age, while subcutaneous fat generally decreases with age. We hypothesized that genetic association studies of a young adult population (average age 24 yrs) would more easily uncover the genetics of adiposity, and initially focused on a study of subcutaneous fat volume.

Obesity is a complex issue with multiple genetic, environmental and behavioral variables as well as age of assessment contributing to the final phenotype. Based on twin and family studies, it is estimated that the heritability of obesity-related phenotypes ranges between 60% and 90%¹. There are well-characterized disease syndromes that include obesity as part of the syndrome (e.g. Prader-Willi). However, the genetic contribution to obesity in the general population is likely the sum of diverse interactions among multiple genes, each contributing a relatively small part to total variation. Each of these adiposity genes may also show different gene-gene and gene-environment interactions, leading to a very complex environmental/genetic network resulting in the final obese phenotype. The identification of markers associated with this common polygenic form of obesity may allow early diagnosis of at-risk individuals and may lead to targeted pharmacological or life style interventions.

As described in the 2004 update of the human obesity gene map, more than 600 genes, markers, and chromosomal regions have been linked to human obesity, with 358 positive association studies linking various obesity related phenotypes to polymorphisms in obesity candidate genes (Perusse, L et al. Obes Res 2005,3:381).

Despite this large number of studies, there are only 18 genes that have been found to be associated with obesity phenotypes in at least 5 different studies, and even for these 18 genes some of the reports have shown opposite findings or negative associations. For example LEPR (Q223R) has been associated with obesity related phenotypes in postmenopausal females (Quinton N D et al. Hum Genet 2001, 3:236)², young adult Dutch (van Rossum C T et al. Obes Res 2001, Obes. Res. 3:377), Caucasian males (Chagnon Y C et al. J Clin Endo Metab 2000, 1:29), obese postmenopausal females (Wauters M et al. Int J Obes Relat Metab Disord 2001, 5:714), and young Greek subjects (Yiannakouris N et al. J Clin Endo Metab 2001, 9:4434), while other studies failed to find associations between the polymorphism and traits related to obesity in Australian women (de Silva A M et al. Obes Res 2001, 12:733) and white British males (Gotoda T et al. Mol Genet 1997, 6:869).

There are many reasons for difficulty in replication of genetic association studies in adiposity. First, there are many different methods of measuring both regional and total adiposity, and each of these methods has well-documented issues concerning reliability of measure, sensitivity, and specificity for measuring fat. Skin folds and MRI imaging can both be used to measure subcutaneous fat, but show different reliability and variation (e.g. triceps vs biceps skinfolds). Second, different studies frequently use different phenotypic measures to characterize adiposity. BMI is often used to provide an “obese” diagnosis to individuals, yet this does not distinguish between fat, muscle and bone content, and is a relatively blunt diagnostic tool. Third, the genetic polymorphisms contributing to obesity may be different across ethnic groups. Indeed, the relationship between body fat percentage and BMI differs among ethnic groups³. Finally, there are often many known or potential confounding factors that may differ in each subject cohort. For example, adiposity in an aged or type 2 diabetes populations may be more subject to uncontrolled confounding variables such as activity, diet, or health problems. A younger normal volunteer population may be less subject to similar confounders, and thus may be more sensitive in uncovering genetic factors.

The genetic underpinnings of adiposity are undoubtedly highly complex. There are likely to be different genetic loci and risk factors for adiposity for specific regions of the body, with sex-specific effects, age-related effects, and ethnicity effects. Indeed, most genetic associations with adiposity have been in elderly or diseased populations, yet it is also known that the genetic factors in an aged population are different than those in a younger population. For example, a longitudinal twin study has reported that only 40% of the genetic factors affecting BMI are shared at the age of 20 y and 40 y (Thompson P D et al. Med Sci Sports Exerc. 2004 7: 132). Moreover, the contribution of a polymorphism to a phenotype may be masked or unmasked by environmental variables or even by polymorphisms in other genes.

We hypothesized that studying young adult healthy volunteers would reduce the effect of many confounding variables which develop with aging. Furthermore, we felt that the use of volumetric MRI for subcutaneous fat volume using specific anatomical landmarks would provide a highly reliable and quantitative measure of regional adiposity.

### SUMMARY OF THE INVENTION

We have invented a test that is prognostic for an increased likelihood of increased regional adiposity (subcutaneous fat) and Body Mass Index (BMI) in Caucasian males, but not females, comprising a determination of the level of a L162 V allelic SNP of the PPARα gene, wherein a statistically significant increase of this allele relative to that of wild-type homozygotes indicates an increased risk for regional adiposity and increased BMI.

In one embodiment of the invention, the increased levels of the L162 V allele predicts an increased likelihood of developing diseases associated with such gene-linked changes in regional adiposity and BMI, such as type 2 diabetes and metabolic syndrome.

In another embodiment of the invention, the absence of statistically significant decreases in the L162 V allelic SNP of PPARα predicts that the subject is unlikely to have increased regional fat and BMI, and a lower expectation of type 2 diabetes and metabolic syndrome.

In yet another embodiment, the absolute level of increased L162 V allelic polymorphism in the subjects PPARα gene is prognostic of the severity of the metabolic consequences of increases in regional subcutaneous fat and BMI.

In still another embodiment, the increased levels of the L162 V allele can be used to judge the effectiveness of drugs and physical exercise that target obesity.

### FIGURE LEGENDS

FIG. 1. shows Pearson correlation between baseline and post-training subcutaneous fat in the untrained arm of the entire cohort. The correlation coefficient between baseline and post exercise subcutaneous fat volume in the untrained-arm was R^{2=0.943} (P=0.001).

FIG. 2. shows correlation between baseline subcutaneous fat volume and baseline body mass index. A strong correlation between BMI and regional arm subcutaneous fat was found in our young adult population: females R²=0.757; P<0.001; males R²=0.661; P<0.001; whole cohort R² 0.543; P<0.001.

FIG. 3. PPARα L162 V is associated with subcutaneous fat in men. Associations between PPAR alfa (L162 V) genotype and baseline subcutaneous fat volume in the trained and untrained arms of males. Panels A & B: age-adjusted models.

### DETAILED DESCRIPTION OF THE INVENTION

We present here the results of a volumetric Magnetic Resonance Imaging (MRI) study of subcutaneous fat volume and BMI in 440 young adult volunteers (average age 24). Fat volume was determined using semi-automated three-dimensional reconstruction analysis software, with anatomical location specified by the epiphyseal flare of the humerus. Fat volume was measured for both upper arms, both before and after a 12 week supervised resistance training program implemented only on one arm. Reliability of measures was determined using the untrained arm, with fat volume measured at a 12 wk interval, showing that our quantitation of subcutaneous fat was highly reliable and sensitive (R²=0.943). Both baseline fat volume and change in fat volume following the training were quantified. We tested 13 polymorphisms in 9 genes that had been previously associated with measures of adiposity (BMI, % fat mass, body fat mass, skin fold thickness, waist circumference), against our volumetric MRI measures. We found strong statistical support for an association of a specific polymorphism in the PPARα gene with increased regional adiposity and BMI. We have discovered, however, that this association is gender- and ethnicity-dependent.

MRI was done for both upper arms before and after 12 wk unilateral supervised resistance training, with subcutaneous fat volume calculated using semi-automated analysis from an anatomical landmark. We tested 13 polymorphisms from 9 genes for association with baseline fat volume, and also for changes in fat volume after training. Each of these candidate loci had been previously shown to be associated with some other measure of adiposity. We found a strong correlation between BMI and regional arm subcutaneous fat in our young adult population (females R²=0.757; P<0.001; males R²=0.661; P<0.001). Eight of the nine genes tested showed no strong association with regional subcutaneous fat volume in young adults in either sex for the ACE, AGRP, ADRβ1, ADRβ2, ADRβ3, LEPR, and TNFα genes. We found strong statistical support for only one gene as a quantitative trait locus (QTL) for subcutaneous fat: PPARα (L162 V). The PPARα (peroxisomal proliferator activated receptor alpha) genotype was associated with baseline subcutaneous fat in Caucasian males (p=0.002; n=113; ancestral 162 L allele; lower fat), but not females. Male heterozygotes for this change showed a 48.5% to 58% increase in subcutaneous fat relative to wild-type homozygotes, and explained 7.0-9.3% of all population variation in subcutaneous fat volume in Caucasian male young adults. The valine allele was associated with increased BMI in Caucasian males (p=0.002) (CC genotype=24.6, CG genotype=28.0). A PPARα polymorphism, L162 V, was found to be strongly associated with subcutaneous fat and BMI in Caucasian males, but not females. This polymorphism did not contribute to adiposity in African-Americans or Asians due to very low allele frequency. Detection of PPARα L162 V polymorphism is thus prognostic for regional obesity, increased BMI, and increased likelihood of serious medical consequences, but only in Caucasian males.

### EXAMPLE 1

### Material and Methods

Study overview and subjects: The Functional Single Nucleotide Polymorphism Associated with Human Muscle Size and Strength or FAMuSS is a multicenter, NIH funded study designed to identify genetic factors that dictate baseline bone, muscle and fat volume and the variability in response to exercise training. The study design protocol has been described in detail elsewhere (Thompson P D et al., above), and preliminary reports of genetic associations with muscle strength and size have been reported (Hubal M J et al. Med Sci Sports Sci. 2005 6:964; Gordon E S et al. Eur J Hum Genet. 2005 9:1047; Clarkson P M et al. J appl. Physiol. 2005 99(1):154). Briefly, 945 men and women, average age 24 (range 18-40 yrs) were recruited by one of the 8 centers (University of Massachusetts Amherst, University of Connecticut, Dublin University (Ireland), Florida Atlantic University, Hartford Hospital, University of Central Florida, West Virginia University, Central Michigan University). Participants were excluded if they: were <18 y or >40 y; used medications known to affect skeletal muscle such as corticosteroids; had any restriction of activity; had chronic medical conditions such as diabetes; had metal implants in arms, eyes, head, brain, neck, or heart which would prohibit MRI testing; had performed strength training or employment requiring repetitive use of the arms within the prior 12 months; consumed on average >2 alcoholic drinks daily; or had used dietary supplements reported to build muscle size/strength or to cause weight gain such as protein supplements, creatinine, or androgenic precursors. Subjects were asked to maintain their normal dietary intake for the duration of the study. Informed consent was obtained from each volunteer. The study was approved by the institutional review board of each participating institution.

Magnetic resonance imaging data (MRI) methods are described below, and complete measurements were available for 440 subjects. Thus, the studies of regional fat volume reported here were limited to these 440 subjects.

Exercise Training Program: Resistant training was performed with the non-dominant arm. The protocol was described elsewhere (Thompson P D et al., above). Briefly it consisted of two 45-60 minute sessions per week for 12 weeks. Each session was supervised by an exercise physiology professional or graduate student. Before each session, participants warm up with 2 sets of 12 repetitions of the biceps preacher curl and the seated overhead triceps extension. Each session included dumbbell biceps curls, dumbbell biceps preacher curls, and incline dumbbell biceps curls, overhead dumbbell triceps extension, and dumbbell triceps kickbacks. The amount of weight was aggressively increased during the 12 wks.

Subject Phenotyping:

Anth]ropometric assessment: Body weight was recorded before and after the exercise training using a balance beam scale. Height was measured using a tape mounted on a wall and recorded in inches. BMI was calculated from weight (kg) and height (m). BMI range was 16.26 to 48.81 for females and 15.50 to 43.75 for males. Average BMI did not differ among the 8 different centers.

MRI assessment: Subjects were scanned in the supine position with arms at their sides and their palms up on the scanner bed surface. The arm maximum circumference was determined with the subject standing, with the shoulder abducted at 90 degrees, the hand supinated, and the biceps flexed. A vitamin E bead was placed on the front of the biceps portion of the arm with the largest circumference to standardized MRI measurements by comparing the bead's measured cross sectional area with that of the MRI determined cross sectional area.

Entry MRI was done 24 to 48 h before the isometric or 1 RM (1 repetition maximum) test. Scans Post-traini]ng MRI was performed 48-96 h after the last training session. Fifteen 16 mm contiguous axial slices from each arm were taken from each arm independently. The top of the bead in a sagital scout view was used to locate the 8^{th} slice going from the top of the arm toward the elbow. Scans for both arms were taken by Fast Spoiled Gradient Recalled (FSPPGR) and Fast Spin Echo (FSE) with TE 1.8/TR 200 msec. All 8 centers submitted the MRI data to the Research Center for Genetic Medicine at Children's National Medical Center (CNMC) in Washington, D.C., via e-mail or DAT disc, and all scans were integrated into the study SQL database.

For both cross-sectional and volumetric analysis of the MRI images, we used Rapidia (INFINITT Inc, Seoul Korea), a PC based software that allows the semi-automatic quantification of muscle, bone and subcutaneous fat. The software was optimized to distinguish muscle, fat, and bone, with automatic edge detection, user modification of ambiguous edges, and automated propagation of defined tissue boundaries when possible. Pre- and post-training volume measures were taken using an anatomical landmark. For this purpose, we defined the metaphyseal-diaphyseal junction of the humerus as our starting point and assayed the six slices proximal to it. The analysis began with the most distal slice and proceeded in a proximal direction until the metaphyseal flare disappeared, using the next six images to make volume calculations. Subcutaneous fat, bone and muscle were isolated using the signal intensity differences between tissues. Semi automatic segmentation tools were used to separate muscle from the overlying fat. Measurement values were automatically written and saved in a SQL database together with anthropomorphic (weight, height, and BMI) and genotyping data.

DNA extraction and genotyping: DNA was extracted from blood samples obtained by phlebotomy before starting the exercise training. Genotyping was done using the TaqMan allele discrimination assay that employs the 5′ nuclease activity of Taq polymerase to detect a fluorescent reporter signal generated during PCR reactions. Standard oligonucleotide primers were used for these assays.

Statistical analyses: The Hardy-Weinberg equilibrium was determined for each SNP using a x² test to compare the observed genotype frequencies to those expected under H-W equilibrium.

Three volumetric measurements were analyzed as continuous quantitative traits (baseline and post-exercise subcutaneous fat volumes and difference in subcutaneous fat volume from baseline to post exercise) for each arm. Normality of each quantitative trait was confirmed using the Shapiro-Wilk normality test.

Bivariate correlation analyses of each quantitative measurement showed several significant correlations with age and baseline mass, therefore, associations between each SNP and volumetric measurements were assessed using analysis of covariance (ANCOVA) methods. Due to the strong relationship between body mass and subcutaneous fat measures, three different modeling schemes were used. These included an age-adjusted model, an age- and weight-adjusted model, and an age-adjusted, BMI stratified model. Due to large gender differences in baseline values and the response to training, all analyses were performed separately for males and females. All significant associations from the main ANCOVA model were subjected to pair-wise statistical tests among each of the three genotype groups for each SNP. Linear tests were performed between each of the genotype groups to determine which genotype groups were significantly different from one another. The resulting p-values from these linear tests were adjusted for multiple comparisons using the Sidak post-hoc multiple comparison test. Linear regression analysis, including likelihood ratio tests between full (containing genotype and covariates) and constrained (containing covariates only) models, were performed to estimate the proportion of variance in volumetric measurements attributable to each SNP's genotype.

### EXAMPLE 2

### Subcutaneous Fat Volume in 440 Young Adult Volunteers

We recruited 945 subjects into a phenotyping and unilateral arm resistance training intervention (“FAMuSS” cohort). Eight sites participated in the study, and all data was entered remotely via a web SQL study database maintained at Children's National Medical Center. Of the 945 initially enrolled, 797 completed initial DNA sampling and phenotyping, and allele frequencies are based on this subset (n=797). 440 subjects completed the intervention and had completed volumetric MRI quantitative measures, and genotype×phenotype associations are based upon this subset (n=440) (Table 1).

TABLE 1
Demographic characteristics of the study population.

| Characteristic | Females (N = 267) | Males (N = 173) |
|---|---|---|
| | N (%) | N (%) |
| Ethnicity | | |
| African-American | 14 (82.4%) | 3 (17.6%) |
| Asian | 20 (40.8%) | 29 (59.2%) |
| Caucasian | 210 (63.4%) | 121 (36.6%) |
| Hispanic | 15 (65.2%) | 8 (34.8%) |
| Other | 8 (40.0%) | 12 (60.0%) |
| | Mean ± SD | Mean ± SD |
| Age (years) | 23.82 ± 5.92 | 25.49 ± 5.85 ** |
| Baseline body mass (lbs.) | 142.49 ± 28.36 | 172.99 ± 34.18 * |
| Baseline height (inches) | 64.78 ± 2.71 | 69.71 ± 3.09 * |
| Baseline body mass index | 23.85 ± 4.44 | 24.98 ± 4.45 ** |
| Post-exercise body mass | 143.28 ± 28.92 | 174.06 ± 34.22 * |
| (lbs.) | | |
| Post-exercise body mass | 23.95 ± 4.57 | 25.11 ± 4.41 ** |
| index | | |

Significantly different means among genders (* p < 0.001; ** p < 0.010)

Regional subcutaneous fat volume of both upper arms, before and after the unilateral strength intervention, was done using a semi-automated image analysis program, Rapidia®. An anatomical landmark (epypheseal flare) was used as the starting point, and 6 slices were analyzed proximal to it. Semi-automatic segmentation tools in the software were used to separate muscle from overlying fat, and muscle from bone.

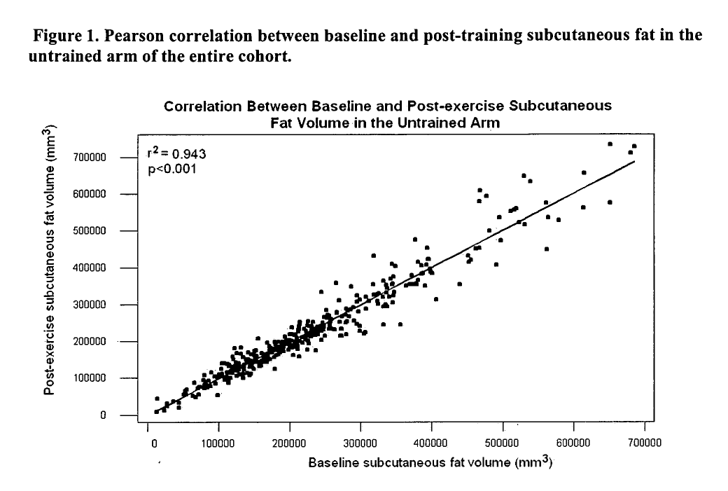

Reliability of measures of subcutaneous fat volume was determined by comparing MRI measurements of the un-trained (dominant) arm at study entry, and again 12 wks later at study exit. We assumed that the un-trained (control) arm would not show significant differences in fat volume over 12 wks, however this can be considered a relatively stringent estimate of measure reliability as this was not adjusted for any weight gain or loss over the 12 wks. The correlation coefficient between baseline and post exercise subcutaneous fat volume in the untrained-arm was R²=0.943 (P=0.001), showing that our quantitation of subcutaneous fat was highly reliable and sensitive (FIG. 1).

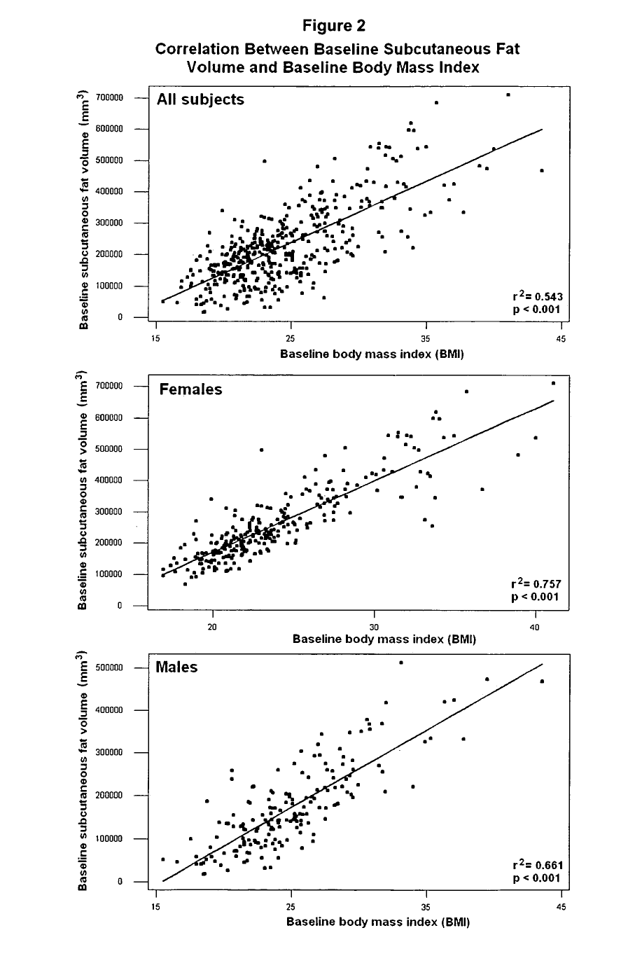

We found a strong correlation of subcutaneous fat volume with BMI (females R²=0.757; P<0.001; males R²=0.661; P<0.001) (FIG. 2). This correlation is higher than expected, as BMI is considered more reflective of central adiposity rather than regional subcutaneous fat.

We considered six phenotypes for association with genetic polymorphisms: absolute fat volume baseline (dominant and non-dominant arms), absolute fat volume after unilateral strength training intervention (both dominant, and non-dominant [trained] arms), and absolute change in fat volume (both dominant and non-dominant arms). All data were stratified for sex (Table 2). Once significant gene×volume associations were identified, the data were further stratified for ethnicity.

TABLE 2
Mean ± SD values for subcutaneous fat volume in our population (all ethnic groups).

| Subcutaneous | | Females | | Males | |
|---|---|---|---|---|---|
| fat volume | Arm | N | Mean ± SD | N | Mean ± SD |
| Baseline | Trained | 267 | 259710.9 ± 117576.3 mm³ | 173 | 172097.9 ± 99036.7 mm³ |
| Post-exercise | Trained | 267 | 263517.7 ± 124380.7 mm³ | 173 | 173123.8 ± 100069.0 mm³ |
| Difference | Trained | 267 | 2636.5 ± 31115.8 mm³ | 173 | 1315.4 ± 23577.6 mm³ |
| Baseline | Untrained | 267 | 262590.0 ± 121448.6 mm³ | 173 | 174873.2 ± 104802.3 mm³ |
| Post-exercise | Untrained | 267 | 263032.0 ± 126839.7 mm³ | 173 | 176217.9 ± 107637.0 mm³ |
| Difference | Untrained | 267 | 415.9 ± 20675.3 mm³ | 173 | 1076.18 ± 29711.6 mm³ |

### EXAMPLE 3

### Genotype Associations with Subcutaneous Fat Volume for 15 Candidate Loci

To identify QTLs for regional subcutaneous fat, 13 polymorphisms in 9 candidate genes already known to be associated with some measure of body fatness were tested in our sample: ACE (I/D; rs17230355)(Strazzulo P et al. Ann Int Med 2003 138:17, AGRP (Ala67Thr; rs5030980)(Argyropolous G et al. J Clin Endo Metab 2002 87:4198), ADRβ1 (Gly389Arg; rs1801253)(Dionne M J et al. Int J Obes Relat. Metab Disord, 2002; 26:633), ADRβ2 (Arg16Gly; rs1042713)(Ishiyama-Shigemoto S et al. Diabetologia 199998); (Gln27Glu; rs1042714)(Meirhaeghe A et al. Int J Obes Rlat Metab Disord, 2000,3:382), ADRβ3 (Arg64Trp; rs4994)(Thomas G N et al. Int J Obes Relat Metab Disord. 2000. 23:545); LEPR (Q223R; rs1137101) (Yannakouris N et al. above); (K109R; rs1137100)(Chagnon Y C et al. above); (Pro1019Pro; rs1805096)(de Silva A M et al, above); (Lys656Asn; rs81179183 (Wauters M et al., above); PAI-1 (−675 4G/5G; rs1799768)(Bouchard L et al. Menopause, 2005 2:136), PPARα (L162 V; rs1800206)(Evans D et al. J Mol Med 2001 4:198); and TNFα (G308A)(Brand E et al. Int J Obes Relat Metab Disord 2001 4:581). All genotyping was done in 96 well plates using highly accurate TaqMan assays, with automated allele calling. Any genotypes considered ambiguous were given a “no call” by the software. Any 96 sample plate showing >7 “no calls” was re-tested.

All loci were genotyped in 797 subjects, and allele frequencies were calculated on this entire group. However, complete MRI data was available for 440 of these subjects, and genetic association studies with adiposity limited to these 440 subjects. Genotype distributions by ethnicity for the large cohort are provided in Table 3.

TABLE 3
Distribution of SNP genotypes among ethnic
groups (n = 797 genotyped subjects):

| | | Total | African- | | | | |
|---|---|---|---|---|---|---|---|
| SNP | Genotype | cohort | Americans | Asians | Caucasians | Hispanics | Others |
| PAI-1 | 4G4G | 175 | 2 | 11 | 153 | 4 | 5 |
| | 4G5G | 337 | 11 | 37 | 262 | 13 | 14 |
| | 5G5G | 195 | 23 | 12 | 139 | 12 | 9 |
| HWE * | | 0.696 | 0.999 | 0.385 | 0.683 | 0.999 | 0.999 |
| LEPR | AA | 239 | 12 | 13 | 187 | 17 | 10 |
| | GA | 419 | 13 | 15 | 349 | 23 | 19 |
| | GG | 216 | 16 | 37 | 148 | 9 | 6 |
| HWE | | 0.586 | 0.241 | 0.027 | 0.910 | 0.999 | 0.889 |
| PPAR | CC | 702 | 39 | 62 | 526 | 42 | 33 |
| | CG | 87 | 1 | 1 | 76 | 8 | 1 |
| | GG | 8 | 0 | 0 | 8 | 0 | 0 |
| HWE | | 0.255 | 0.999 | 0.999 | 0.247 | 0.812 | 0.999 |

* HWE - p-value is presented

We found most loci to be in Hardy Weinberg Equilibrium (HWE), with the single exception of the LEPR polymorphism in Asians. We attribute this deviation from Hardy Weinberg Equilibrium to the relatively low number of subjects (n=65), and relatively ambiguous patient-reported nature of “Asian”.

Genotype/phenotype associations were done as continuous quantitative variables using ANOVA, with three genotype groups per locus considered as separate groups (AA, AB, BB). All data were stratified by sex. Age was treated as a co-variant with subcutaneous fat volume, despite the relatively narrow age distribution of our cohort, and all data was statistically adjusted for age. As expected, both BMI and weight were seen to be strong co-variants with subcutaneous fat. Each locus was tested without weight adjustment and with weight adjustment (Table 6) and with stratification by BMI (<25, and 25) (not shown; generally similar to weight stratification).

Considering genotype associations without weight adjustment, three of the 13 SNPs studied, AGRP (Ala67Thr), LEPR (Q223R), and PPARα (L162 V) showed statistically significant association with one or more absolute measures of subcutaneous fat volume in men (Table 4). None of these showed significance in females. PAI-1-675 4G/5G was associated with change in subcutaneous fat volume in response to exercise training in women; this did not show significance in men.

The strongest and most significant association was seen for the PPARα gene, and this was studied in greater detail (see below). The statistical associations with AGRP, LEPR, and PAI-1 were less compelling (Table 4).

TABLE 4
Percent variability of subcutaneous fat volume predicted
by LEPR Q223R, PPARα L162V, AGRP and PAI-1 4G5G.

| | | | | | | Variability |
|---|---|---|---|---|---|---|
| | | Phenotype | Statistical | N: adjusted | Ancestral | attributable |
| SNP | Gender | Subcu. fat volume | model | mean ± SEM | allele | to genotype |
| | | | | AA (N = 25; 285296 ± 17988) * | A allele | |
| LEPR Q223R | Male | U. arm baseline | BMI > 25 | AG (N = 35; 229458 ± 15373) | | 12.80% |
| | | | | GG (N = 13; 194606 ± 24320) * | | |
| PPAR L162V | Male | T. arm baseline | Full model | CC (N = 95; 160449 ± 9762.8) * | L allele | 7.60% |
| | | | | CG (N = 18; 23882 ± 22400.5) * | | |
| | | T. arm post | Full model | CC (N = 95; 162025 ± 10079.3) * | | 7.10% |
| | | | | CG (N = 18; 239866 ± 22999.9) * | | |
| | | U. arm baseline | Full model | CC (N = 95; 163097 ± 10405.3) * | | 7.50% |
| | | | | CG (N = 18; 246531 ± 23874.8) * | | |
| | | U. arm post | Full model | CC (N = 95; 162106 ± 10510.6) * | | 9.30% |
| | | | | CG (N = 18; 256388 ± 24116.5) * | | |
| PAI-1 4G5G | Female | T. arm difference | BMI > 25 | 4G5G (N = 32; 14839 ± 7969) * | 5G allele | 10.70% |
| | | | | 5G5G (N = 20; −19511 ± 10389) * | | |
| AGRP A67T | Male | T. arm baseline | Full model | GA (N = 10; 235932 ± 31398) | | 2.60% |
| | | | | GG (N = 148; 169453 ± 8171 | | |

The AGRP associations were significant for three different baseline measures in males, however each showed borderline significance. The associations were opposite from previously reported associations: a previous report showed Caucasians older than 50 yrs with the GG genotype to show higher adiposity, while we found this same genotype associated with lower adiposity in males only (Table 4). The LEPR polymorphism showed significance with baseline fat volume in males, however this association was driven by Asian males, with significance lost when testing Caucasians alone. The Asian males were low in number (n=16), and the allele distributions were not in Hardy Weinberg Equilibrium within this subgroup. Thus, further study would be required to determine the significance of LEPR genotype within the Asian subpopulation. PAI-1 showed significant association with change in subcutaneous fat volume with training in females.

This was significant only in the trained arm, and not the un-trained arm, leading to the possible image interpretation differences in the trained and untrained arm. Also, the significance was found only for heterozygotes, where homozygotes for both the common and rare alleles showed lower changes relative to heterozygotes. Thus, a plausible biochemical model for the effect was difficult to define. Finally, the p values were borderline (p=0.03) only in Caucasians. For these reasons, we focused on the more robust statistical associations found with PPARα.

### EXAMPLE 4

### Characterization of Associations with PPARα Polymorphism

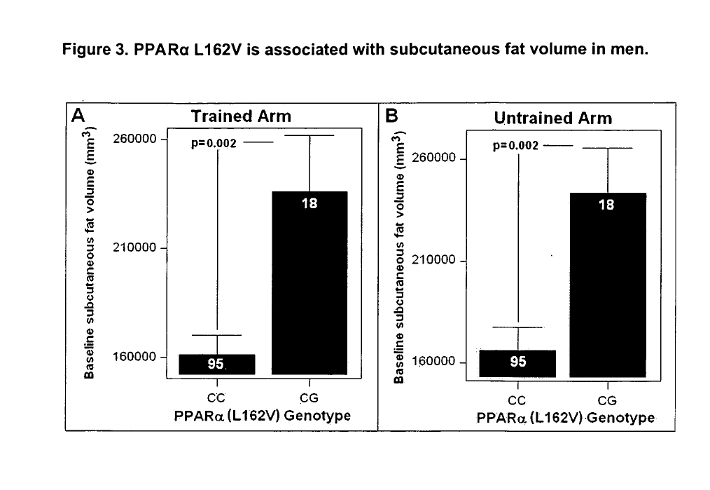

The allele frequencies of the PPARα L162 V allelic polymorphism showed this rare valine allele at 7.5% in Caucasians, with 18% of Caucasians heterozygous for the allele. Both Asians and African Americans showed very low allele frequencies; this was likely due to admixture in the college population studied, particularly as ethnicity was self-reported by the volunteers. The PPARα 162 valine allele was associated with significantly increased baseline regional subcutaneous fat volume in Caucasian men in the trained arm, and showed similar significance in all four absolute measures (trained, un-trained, entry and exit; FIG. 3; Panels A, B.

Male heterozygotes for L162 V showed 48.5% to 58% higher regional subcutaneous fat than homozygotes for the common ancestral allele (CC genotype=162, 106 mm³; CG genotype=256,388 mm³). As 18% of the population carried the rare allele, the genotype effect of the SNP for all variation in regional subcutaneous fat volume is males was approximately 7.0-9.3%.

For female subjects, our analysis did not show any statistically significant difference for the studied phenotype in the presence of the variant allele. Similarly, no significant differences were seen in response to exercise for both females and males. We also queried association between PPARα and BMI in Caucasian males (as an independent variable). The valine allele was associated with increased BMI in Caucasian males (P=0.002) (CC genotype=24.633, CG genotype=27.97) (n=113).

In summary, those Caucasian males carrying the L162 V allele have greater regional subcutaneous fat volume, and are more likely to be overweight as measured by BMI. It is also concluded that the greater the concentration of this allele in this ethnic/gender group, the greater the likelihood of increased regional fat and BMI, and the greater the likelihood of metabolic sequel such as type 2 diabetes and metabolic syndrome. Conversely, the absence of statistically significant increases in the L162 V allele, makes it the less likely that the subject will acquire increases in regional fat and BMI and to acquire type 2 diabetes and metabolic syndrome. The inventive test can be used to design treatment regimens for patients who are genetically likely to acquire regional adiposity and increased BMI.

### EXAMPLE 5

### Conclusions

We carried out a genetic association study of BMI and upper arm subcutaneous fat volume, before and after a 12 wk unilateral resistance training in young adult volunteers (average age 24 yrs). This cohort included 797 subjects phenotyped for BMI and strength data, although quantitative MRI for subcutaneous fat, before and after the intervention, was available for 440 of these. We tested previously reported QTLs for obesity-related phenotypes and compared the genotypes to subcutaneous fat volume of both upper arms, and BMI. The subcutaneous fat phenotype was measured by analyzing volumetric MRI images using a highly reliable (R²=0.943) semi-automated three-dimensional analysis software. We found a strong correlation between BMI and regional arm subcutaneous fat in our young adult population (females R²=0.757; P<0.001; males R²=0.661; P<0.001).

We tested 13 loci in 9 genes for association with either BMI or subcutaneous fat volume (n=440). Each of these genes had been previously reported to show statistical association with some measure of adiposity or obesity. In general, our data in young adult volunteers was not concordant with previous reports, in that the majority of loci showed no significant association with either BMI or subcutaneous fat.

We found strong statistical support for association of a PPARα polymorphism (L162 V) with subcutaneous fat volume and BMI in Caucasian males. We found that Caucasian male carriers of the 162 V allele showed significantly increased regional subcutaneous fat volume (n=113; p=0.002), and genotype at this locus explained approximately 7.1-9.3% of all variation in subcutaneous fat. Male heterozygotes for this change showed a 48.5 to 58% increase in subcutaneous fat relative to wild-type homozygotes. Caucasian male carriers also showed a significantly increased BMI (n=113; p=0.002). These findings and their relationship to previous reports and the biological activity of PPARα are discussed in depth below. Of the other 8 genes, three genes showed borderline statistical significance (AGRP, PAI-1, LEPR), two were significant only when statistically adjusting for weight (ADRB1 and ADRB2), and three loci showed no significance with any subcutaneous fat measure (ACE, ADRβ3, and TNFα).

Unexpectedly, PPARα valine allele showed strong statistical significance with increased subcutaneous fat and BMI in young Caucasian males in our study; the two previous reports showed association of the valine allele with decreased percentage of body fat, and lower BMI, in overweight and diabetic females (Evans D et al., above; Brand E et al.) The differences between the results of Evans et al and those of our study may be explained by the major differences in the cohorts studied. The association found in their study was restricted to diabetic females or morbidly obese males and females, suggesting that the reported effect of the polymorphism is not present in healthy individuals and could be dependent on diabetic state or on high BMI. We studied young healthy individuals with less confounding factors than older or diseased subjects.

Bosse et al. (Obes Res. 2003 7:809) have reported association of heterozygotes (162 V) and lower BMI (P=0.023) and percentage body fat by hydrostatic weighing (P=0.026) in healthy volunteers (n=698; mean age 44, mean BMI 27). When their cohort was stratified by sex the association was no longer significant in either sex. The authors did not find statistically significant differences in abdominal fat volumes measured by computed tomography (total, subcutaneous, and visceral cross sectional area). In our cohort of 797 healthy volunteers, we found the 162 V allele associated with increased BMI in Caucasian males, and this is clearly in contrast to the decreased BMI in males plus females reported by Bosse et al, above. The key distinction may be the age groups studied (24 yrs vs 44 yrs average). Given that it has been reported that only about half the genetics of obesity in 20 yr olds is shared with genetics of obesity in 40 yr olds, our studies may define PPARα as one of the differences between these age groups.

PPARα is a member of the nuclear hormone receptor superfamily that controls the expression of genes involved in glucose and lipid homeostasis. The receptor is activated by circulating fatty acids, especially polyunsaturated fatty acids and, peroxisome proliferators receptor agonists such as hypolipidemic drugs (fibrates), commercially used plasticizers, synthetic fatty acids, steroid hormones, herbicides and pesticides. PPARα is highly expressed in tissues with a high ratio of fatty acid oxidation such as liver, skeletal and cardiac muscle and kidney. Activation of the receptor results in the increased expression of genes involved in lipid oxidation (Acyl-CoA synthetase, Carnitine palmitoyltransferase-I), lipoprotein metabolism (Lipoprotein lipase, Apo A-I, Apo A-II and inhibition of Apo C-III), inhibition of vascular inflammation and adipocyte differentiation through binding to peroxisome proliferator response elements (PPRE) in the DNA sequence of target genes. In summary the activation of PPARα stimulates fatty acid transportation and oxidation primarily in liver and muscle reducing the storage of fat adipocytes. Studies in mice have shown that PPARα-deficient animals were unable to metabolize lipids and develop late onset obesity even when kept on a stable diet.

The L162 V polymorphism is a missense mutation in exon 5 of the receptor gene that results in a nonconservative amino acid substitution; L162 is located within the DNA binding domain and is highly conserved among species. Sapone et al. (Pharmacogen. 2000 4:321) studied the functional significance of this point mutation using transfection assays. The authors reported higher transactivation of the receptor in the presence of the V allele in a ligand concentration dependant way. At levels lower than 25 μM or total absence of the peroxisome proliferator, the activity of the variant receptor was lower than the wild type. With a concentration >25 μM of the ligand, the activity was clearly higher. Since the activity of the receptor is in part dictated by the concentration of the receptor agonists and PPARα is activated by fatty acids among others, we would expect to see higher activity of the polymorphic receptor in obesity, metabolic syndrome and type 2 diabetes (in which the levels of fatty acids are higher). Levels of endogenous or exogenous ligands (such as dietary fatty acids) may determine the activity of the polymorphic receptor and dictate the final phenotype observed in the presence of this polymorphism.

We claim:

1. A method for predicting regional adiposity and increased BMI in a subject, comprising the steps of obtaining a DNA sample from a subject to be assessed, determining the nucleotide present in position 162 in exon 5 of the PPARα gene of said DNA sample, wherein an increase in L162 V valine allelic single nucleotide polymorphism (“SNP”) in said PPARα gene predicts a likelihood for increased regional adiposity and BMI in said subject, relative to wild-type homozygotes.

2. The method of claim 1, wherein said subject is a Caucasian male.

3. The method of claim 1, wherein said region is subcutaneous fat.

4. A method of predicting regional adiposity and increased BMI in a subject, comprising the steps of obtaining a DNA sample from a subject to be assessed, determining the nucleotide present in position 162 in exon 5 of the PPARα gene of said DNA sample, wherein no statistically significant difference between the level of L 162 V valine allelic SNP, relative to that of wild-type homozygotes predicts the likelihood of no gene-linked increase in regional adiposity and BMI in said subject.

5. The method of claim 4, wherein said subject is a Caucasian male.

6. The method of claim 4, wherein said region is subcutaneous fat.

7. A method for predicting the severity of the symptomology associated with increased subcutaneous fat and BMI in a subject, comprising the steps of obtaining a DNA sample from a subject to be assessed, determining the nucleotide present in position 162 in exon 5 of the PPARα gene of said DNA sample, wherein an increase in L162 V valine allelic SNP in said PPARα gene predicts the likelihood of an increase in the severity of said symptomology in said subject, relative to wild-type homozygotes.

8. The method of claim 7, wherein said symptom is related to type 2 diabetes.

9. The method of claim 7, wherein said symptom is increased metabolic syndrome.

10. A method of targeting individuals more likely to benefit from treatment with anti-obesity drugs or physical exercise comprising the steps outlined in claims 1-3, wherein an individual with a statistically significant increase in the PPARα L162V allele is more likely to benefit from said treatments drugs.
